(19) United States
(12) Patent Application Publication
(10) Pub. No.: US 2026/0315642 A1
(43) Pub. Date: Oct. 15, 2026

(54) UV-C SPIRAL REFLECTOR SYSTEMS AND MEHTODS

(71) Applicant: James Phillip Jones, Sudan, TX (US)

(72) Inventor: James Phillip Jones, Sudan, TX (US)

(21) Appl. No.: 19/641,327

(22) Filed: Apr. 7, 2026

Related U.S. Application Data
(60) Provisional application No. 63/785,866, filed on Apr. 9, 2025.

(51) Int. Cl. A61L 9/20; F24F 8/22

(52) U.S. Cl. CPC A61L 9/20; F24F 8/22; A61L 2209/12; A61L 2209/16

(57) ABSTRACT

A UV-C Spiral Reflector System for efficiently distributing UV light within a HVAC conduit or a static volume to efficiently disinfect the airflow passing through the conduit or in the static volume. A device is presented to be able to modify older systems by inserting the device through the side of the conduit.

### PRIORITY APPLICATIONS

This non-provisional application claims priority from provisional application 63/785,866 to Phillip Jones filed on Apr. 9, 2025.

### FIELD OF THE INVENTION

He present invention relates to at least one device, at least one system, and at least one process or method to be used within the medical sciences, engineering, research, medical and general construction technologies for disinfecting air traveling through Heating, Ventilation, and Airconditioning (“HVAC”) Systems.

### BACKGROUND

Ultraviolet germicidal irradiation (“UVGI”) is a disinfection technique employing ultra-violet (UV) light, particularly UV-C (180-280 nm), to kill or inactivate microorganisms. UVGI primarily inactivates microbes by damaging their genetic material, thereby inhibiting their capacity to carry out vital life functions.

The use of UVGI extends to an array of applications, encompassing food, surface, air, and water disinfection UVGI devices can inactivate microorganisms including bacteria, viruses, fungi including bacteria, viruses, fungi, molds, and other pathogens. Recent studies have substantiated the ability of UV-C light to inactivate SARS-CoV-2, the strain of coronavirus that causes COVID-19.

UV-C wavelengths demonstrate varied germicidal efficacy and effects on biological tissue. Many germicidal lamps like low-pressure mercury (LP-Hg) lamps, with peak emissions around 254 nm, contain UV wavelengths that can be hazardous to humans. As a result, UVGI systems have been primarily limited to applications where people are not directly exposed, including hospital surface disinfection, upper-room UVGI, and water treatment.

More recently, the application of wavelengths between 200-235 nm, often referred to as “far-UVC”, has gained traction for surface and air disinfection. These wavelengths are regarded as much safer due to their significantly reduced penetration into human tissue. Moreover, their efficiency relies on the fact that in addition to the DNA damage related to the formation of pyrimidine dimers, they provoke important DNA photoionization, leading to oxidative damage. Pyrimidine dimers are DNA lesions formed when UV light induces covalent bonds between adjacent pyrimidine bases, disrupting DNA structure and potentially causing mutations. Notably, UV-C light is virtually absent in sunlight reaching the Earth's surface due to the absorptive properties of the ozone layer within the atmosphere.

Due to the harmful physical effects of UVC light, it is desirable to minimize the amount of light used to disinfect a given volume of air by more evenly, and efficiently, irradiating that air.

### SUMMARY

The subject matter presented herein describes a UV system and method to disinfect a volume of air passing thought an air handling conduit/system. Although an air handling system is specifically described herein as a matter of brevity, the concept, systems and methods are similarly applicable to water handling/piping systems with adjustments for the density differences between air and water and the turbidity variations thereof.

The systems described herein use spiral-shaped reflective structures inside a closed or semi-enclosed chamber. These spiral reflectors improve UV light distribution, reducing shadowed areas and increasing overall microbial inactivation efficiency.

### BRIEF DESCRIPTIONS OF THE DRAWINGS

FIG. 1 depicts a first exemplary non-limiting cross sectional/perspective view of an exemplary embodiment of the system in HVAC air duct/conduit.

FIG. 2 depicts a modular UV-C light and spiral shaped reflective structures.

FIG. 3 depicts a second exemplary non-limiting exemplary cross sectional/perspective view of an exemplary embodiment of the system in HVAC air duct/conduit.

### DETAILED DESCRIPTION

The following detailed description is merely exemplary in nature and is not intended to limit the invention, or the application, or uses of the subject matter disclosed. As used herein, the word “exemplary” means “serving as an example, instance, or illustration.” Thus, any embodiment described herein as “exemplary” is not necessarily to be construed as preferred or advantageous over other embodiments. All of the embodiments described herein are exemplary unpowered embodiments provided to enable persons skilled in the art to make or use the invention and not to limit the scope of the invention which is defined by the claims. As used herein, the term “unpowered” means “without a battery” and/or “without AC power.” Furthermore, there is no intention to be bound by any expressed or implied theory presented in the preceding technical field, background, brief summary, or the following detailed description.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

It should be understood that the terms “right” and “left” are used solely to denote opposite sides of an element, component, or surface in the same manner that “top” and “bottom” are used solely to denote opposite sides of an element, component, or surface and should not unnecessarily be construed as limiting the position or orientation of said element, component, or surface.

In the following description, for purposes of explanation, numerous specific details are set forth in order to provide a thorough understanding of the present invention. It will be evident, however, to one skilled in the art that the present invention may be practiced without these specific details.

In this document, relational terms such as first and second, and the like may be used solely to distinguish one entity or action from another entity or action without necessarily requiring or implying any actual such relationship or order between such entities or actions. Numerical ordinals such as “first,” “second,” “third,” etc. simply denote different singles of a plurality and do not imply any order or sequence unless specifically defined by the claim language. The sequence of the text in any of the claims does not imply that process steps must be performed in a temporal or logical order according to such sequence unless it is specifically defined by the language of the claim. The process steps may be interchanged in any order without departing from the scope of the invention as long as such an interchange does not contradict the claim language and is not logically nonsensical.

Furthermore, depending on the context, words such as “connect” or “coupled to” used in describing a relationship between different elements do not imply that a direct physical connection must be made between these elements. For example, two elements may be connected to each other physically, electronically, logically, or in any other manner, through one or more additional elements.

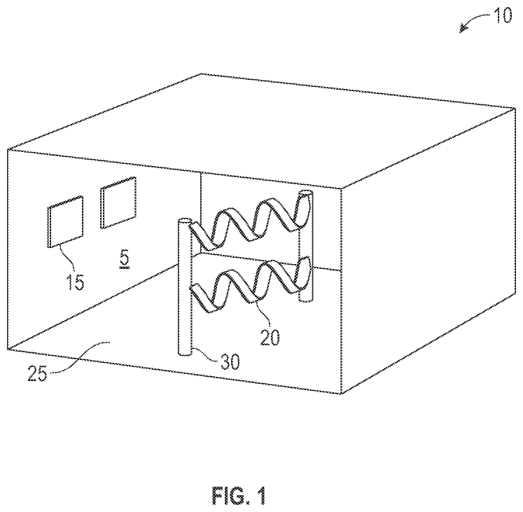

Reference should now be made to the drawings in which the same reference numbers are used throughout the various figures to designate the same or similar components. FIG. 1 is a cross-sectional, perspective view of an exemplary HVAC conduit 10 showing the new and novel UV disinfectant features. The cross-sectional shape of the conduit shown is rectangular as an example but the conduit can be of any cross-sectional shape as may be desired by the designer's system plans and specifications. The internal walls 25 of the conduit/conduit section may have a smooth mirror like finish or may may comprise a textured reflective surface to scatter the UV-C light into all reaches and corners of the conduit. The surfaces may be dimpled or surfaced with Polytetrafluoroethylene. In some exemplary embodiments, the inside walls may not be reflective of UV-C radiation at all.

The exemplary system comprises at least one, or multiple, UV-C light source(s) 15 mounted inside conduit 10 or on the inside wall(s) of the conduit 10. The frequency of the UV-C light/radiation source(s) 15 emit radiation in the germicidal range of approximately 180-280 nm, and preferably in the 265-270 nm range. UV-C light is comprised of photons. The UV-C light sources 15 may be located in the most advantageous locations to flood the entire conduit volume and minimize or eliminate any shadow zones where the UV light may not directly illuminate sufficiently without the aid of the novel spiral or helical reflectors 20. Exemplary, non-limiting reflective materials that tend to withstand degradation in UV light include but are not limited to Aluminum, Silver, Magnesium Floride, or an alloy or combination thereof.

The helical shape of the reflectors continuously change its reflective orientation along the length of the helical or spiral reflector(s) 20. The reflector(s) 20, in conjunction with the reflective inside surface(s) of the conduit, increase the probability that linearly traveling UV-C photons emitted from the light source(s) 15 are reflected many times and cross and re-cross the treatment volume 5 and the fluid inside (not shown) the conduit 10 multiple times thereby improving exposure of micro-organisms in the fluid to the UV-C light. The multiple reflections between the reflectors 20 and the reflective inside surfaces of the conduit 25 allow use of light sources (e.g., a bulb) with lower lamp power or and shorter conduit lengths of travel to achieve the same disinfection results.

The spiral or helical reflectors 20 may be configured as continuous flat ribbons that are thin elongated flat strips of material that are highly reflective on both sides; or, may be configured as elongated strands of highly reflective material having one or more of a circular cross section, an oval cross section, and a polygonal cross section such as a square, triangle or hexagon, for example. The helical/spiral geometry of the reflectors 20 contribute to higher angular distribution of photon paths and increased photon path recycling within the conduit treatment volume 5.

It should be noted that in all embodiments the fluid discussed herein may be a liquid or it may a gas, such as the atmosphere which is ubiquitous and inherent and as such not shown in the drawings. It also should be noted that the fluid may be static in the volume 5 or it may be flowing through the volume 5 while being irradiated as would be the case in a HVAC application.

The exemplary system shown in FIG. 1 includes at least one spiral reflector 20 that is suspended or mounted in the or near an internal axis of the conduit 10. However, this location is and number is not intended to be limiting and the location at which the one or more spiral reflectors are placed may be offset from a centerline axis as may accommodate the shape and design of the HVAC system and minimize or eliminate any shadow zones where microorganisms may congregate in a shadow zone. The spiral reflector mount(s) 30 may be of any shape or configuration as may be desired by the designer and may be powered or may be simple mechanical mounts. The reflector mount(s) 30 may be made of a clear material that let UV-C energy pass through. Exemplary, non-limiting materials that let UV-C energy pass through may include fused silica (UV-Grade Quartz), Magnesium Fluoride (MgF₂), Calcium Fluoride (CaF₂), UV-transmitting acrylic (“PMMA-UVT” or “UV-Transmitting Polymethyl Methacrylate), Cyclic Olefin Copolymer (COC) plastic such as TOPAS COC produced by Daicel Corporation.

The spiral reflectors 20 may be coated with a self-cleaning may include any of a protective or self-cleaning coating that my include Photocatalytic TiO₂, a reflective super hydrophobic material, a dust-repellant material or dielectric coating that enhance such as polytetrafluoroethylene to maximize efficient UV-C bounce.

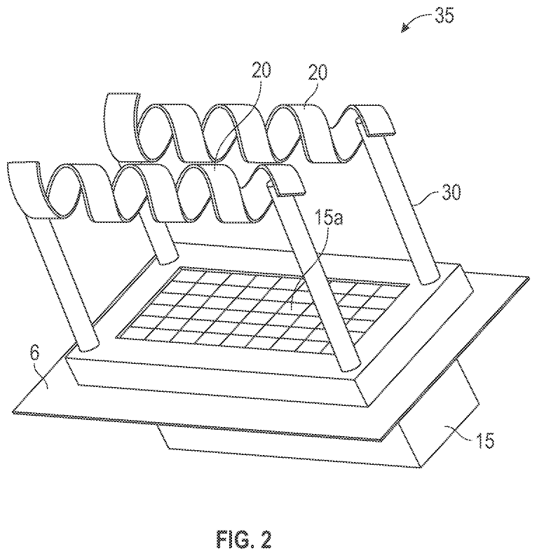

FIG. 2 depicts an exemplary, non-limiting modular UV-C light reflection unit 35 that may be used to install through a corresponding opening in a side of a rectangular fluid conduit such as an HVAC conduit. A UV-C light 15 is constructed through and secured to a plate 6, or other foundational element, with the external portion of the light 15b protruding through the bottom of side plate 6 and the light emitting portion 15a protruding on the internal surface. In this exemplary embodiment there is depicted two parallel helical reflectors 20, each attached to a pair of spiral reflector mounts 30 that may space the helical reflectors 20 away from the UV-C radiation source 15a/15b. The external portion of the light 15b may be connected to an AC or DC power source, or may contain a battery to power the light emitting portion 15a. The spiral ribbon reflectors 20 and the corresponding spiral reflector mounts 30 are inserted through an opening in a side of a conduit 10 and the plate 6 may be removably secured to the side of the conduit.

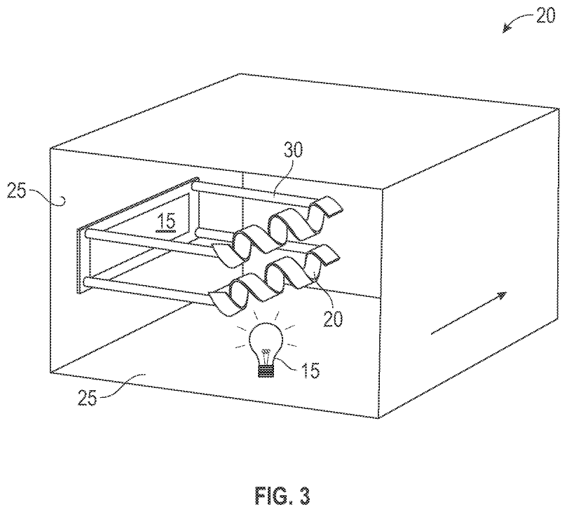

FIG. 3 is a cross-sectional, perspective view of another exemplary, non-limiting HVAC conduit 10 to that of FIG. 1 showing the new and novel UV disinfectant features using UV-C light reflection unit 35. The cross-sectional shape of the conduit shown is also rectangular as an example but the conduit can be of any cross-sectional shape as may be desired by the designer's system plans and specifications. The internal walls 25 of the conduit/conduit section may have a smooth mirror like finish or may may comprise a textured reflective surface to scatter the UV-C light into all reaches and corners of the conduit. The surfaces may be dimpled or surfaced with Polytetrafluoroethylene. In some exemplary embodiments, the inside walls may not be reflective of UV-C light radiation at all.

As disclosed in regard to FIG. 1, the exemplary system of FIG. 3 comprises at least one, or multiple, UV-C light source(s) 15 mounted inside conduit 10 or on the inside wall(s) of the conduit 10 as a component of an UV-C light reflection unit 35. The frequency of the UV-C light source(s) 15 emit radiation in the germicidal range of approximately 180-280 nm, and preferably in the 265-270 nm range. The light sources 15 as part of a UV-C light reflection unit 35 may be located in the most advantageous locations to flood the entire conduit volume and minimize or eliminate any shadow zones where the UV light may not directly illuminate sufficiently without the aid of the novel spiral or helical reflectors 20. Exemplary, non-limiting reflective materials that tend to withstand degradation in UV light include but are not limited to Aluminum, Silver, Magnesium Floride, or an alloy or combination thereof.

By continuously changing its reflective orientation along the length of the helical or spiral reflectors 20 increase the probability that linearly traveling UV-C photons emitted from the light source(s) 15 cross and re-cross the treatment volume 5 inside the conduit 10 multiple times thereby improving exposure of micro-organisms to the UV-C light allowing the use of light sources with lower lamp power or and shorter conduit lengths to achieve the same disinfection results. The spiral or helical reflectors 20 may be configured as continuous flat ribbons that are thin elongated flat strips of material that are highly reflective on both sides; or, may be configured as elongated strands of highly reflective material having one or more of a circular cross section, an oval cross section, and a polygonal cross section such as a square, triangle or hexagon, for example. The helical/spiral geometry of the reflectors 20 contribute to higher angular distribution of photon paths and increased photon path recycling within the conduit treatment volume 5.

The exemplary system shown in FIG. 3 includes at least one spiral reflector 20 that is attached to or dependent from a UV-C light reflection unit 35. The attached spiral reflector 20 can be suspended or mounted in the or near an internal axis of the conduit 10. However, this location is and number is not intended to be limiting and the location at which the one or more spiral reflectors are placed may be offset from a centerline axis as may accommodate the shape and design of the HVAC system and minimize or eliminate any shadow zones where microorganisms may congregate in a shadow zone. The spiral reflector mount(s) 30 may be adjustable in height and of any shape or configuration as may be desired by the designer or may be simple mechanical mounts. The reflector mount(s) 30 may be made of a clear material that let UV-C energy pass through. Exemplary, non-limiting materials that let UV-C energy pass through may include fused silica (UV-Grade Quartz), Magnesium Fluoride (MgF₂), Calcium Fluoride (CaF₂), UV-transmitting acrylic (“PMMA-UVT” or “UV-Transmitting Polymethyl Methacrylate), Cyclic Olefin Copolymer (COC) plastic such as TOPAS COC produced by Daicel Corporation.

As disclosed in regard to the embodiment of FIG. 1, the spiral reflectors 20 may be coated with a self-cleaning may include any of a protective or self-cleaning coating that my include Photocatalytic TiO₂, a reflective super hydrophobic material, a dust-repellant material or dielectric coating that enhance such as polytetrafluoroethylene to maximize efficient UV-C bounce.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

What is claimed is:

1. A system for efficiently disinfecting a fluid in a physically bounded volume comprising:
at least one wall with an inside and an outside surface bounding the volume;
at least one source of UV-C radiation mounted within the volume; and
at least one helical shaped UV-C radiation reflector mounted within the volume.

2. The system of claim 1, wherein the inside surface of the bounded volume is comprised of a material highly reflective of UV-C radiation.

3. The system of claim 2, wherein the UV-C radiation source emits UV-C radiation with a frequency below 300 nm.

4. The system of claim 1, wherein the bounded volume is a conduit transporting a fluid.

5. The system of claim 1, wherein the at least one UV-C radiation reflector mounted in the volume is mounted on at least one reflector mount comprised of a material highly transmissive to UV-C radiation.

6. The system of claim 5, wherein the material highly transmissive to UV-C radiation is comprised of at least one of fused silica (UV-Grade Quartz), Magnesium Fluoride (MgF₂), Calcium Fluoride (CaF₂), UV-Transmitting Polymethyl Methacrylate, and Cyclic Olefin Copolymer (COC) plastic.

7. The system of claim 2, wherein the material highly reflective to UV-C radiation is comprised of at least one of any of Aluminum, Silver, Magnesium Floride or an alloy or combination thereof.

8. The System of claim 1, wherein the at least one reflector is mounted to and spaced a distance away from the source of UV-C radiation by at least one reflector mount.

9. A method for efficiently disinfecting a fluid in a volume, comprising:
providing a physically bounded volume comprising at least one wall with an inside and an outside surface;
providing at least one source of UV-C radiation within the volume;
providing at least one helical shaped UV-C radiation reflector fixedly mounted within the volume and;
irradiating the fluid in the volume with the UV-C radiation, wherein the at least one helical shaped UV-C radiation reflector increases the probability that UV-C photons emitted from the at least one UV-C light source reflect from the UV-C radiation reflector and cross and re-cross the treatment volume multiple times.

10. A device for improving the disinfectant efficiency of UV-C radiation in a fluid, comprising:
at least one source of UV-C radiation mounted to a foundational element; and
at least one helical shaped UV-C radiation reflector attached to the foundational element and spaced a distance away from the source of UV-C radiation by at least one reflector mount.

11. The device of claim 10, wherein the foundational element comprises a power source.

12. The device of claim 11, wherein the power source energizes the source of UV-C radiation causing it to radiate UV-C radiation.
